(19) United States
(12) Patent Application Publication
(10) Pub. No.: US 2022/0151856 A1
(43) Pub. Date: May 19, 2022

(54) DETECTION DEVICE AND WALKING SUPPORT SYSTEM

(71) Applicant: Japan Display Inc., Tokyo (JP)

(72) Inventor: Go KIHARA, Tokyo (JP)

(21) Appl. No.: 17/527,673

(22) Filed: Nov. 16, 2021

(30) Foreign Application Priority Data
Nov. 19, 2020 (JP) 2020-192800

(51) Int. Cl. A61H 3/00; G01L 1/04; F16J 15/06

(52) U.S. Cl. CPC A61H 3/00; F16J 15/061; G01L 1/04

(57) ABSTRACT

According to an aspect, a detection device includes: an elastic body that has a surface part on which a first recess and a second recess are formed and that forms a first design generated on the surface part by the first recess and the second recess according to a first load and a second design generated on the surface part by the first recess and the second recess according to a second load, the first design being different from the second design; and a sensor configured to detect a recess and a protrusion on the surface part.

### CROSS-REFERENCE TO RELATED APPLICATION

This application claims the benefit of priority from Japanese Patent Application No. 2020-192800 filed on Nov. 19, 2020, the entire contents of which are incorporated herein by reference.

### BACKGROUND

### 1. Technical Field

What is disclosed herein relates to a detection device and a walking support system.

### 2. Description of the Related Art

It is known that there are sensors that detect force and sensors that detect deformation caused by force (e.g., Japanese Examined Patent Application Publication No. H6-66478 and Japanese Patent Application Laid-open Publication No. 2001-21308).

There is a demand to deal with the direction of a load, which is applied by a subject (e.g., a walking person) that acts to move in a certain direction in planar view to an object (e.g., a shoe sole) in contact with the subject, as one-dimensional information. The sensors described above, however, are not designed for such use.

For the foregoing reasons, there is a need for a detection device and a walking support system suitable for determining the direction of a load (load direction) applied to an object by an acting subject.

### SUMMARY

According to an aspect, a detection device includes: an elastic body that has a surface part on which a first recess and a second recess are formed and that forms a first design generated on the surface part by the first recess and the second recess according to a first load and a second design generated on the surface part by the first recess and the second recess according to a second load, the first design being different from the second design; and a sensor configured to detect a recess and a protrusion on the surface part.

### BRIEF DESCRIPTION OF THE DRAWINGS

FIG. 1 is a schematic diagram of a shoe including a detection device;

FIG. 2 is a schematic diagram illustrating an exemplary main configuration of the detection device;

FIG. 3 is a plan view illustrating an example of a specific shape of an insole and the difference in size between design-comprising parts;

FIG. 4 is a sectional view illustrating an example of a relation between the design-comprising part and the insole;

FIG. 5 is a schematic diagram illustrating an elastic body in planar view in a state in which no load is applied to the elastic body;

FIG. 6 is a schematic diagram illustrating the elastic body in planar view in a state in which a load Po in a certain direction is applied to the elastic body;

FIG. 7 is a schematic diagram illustrating a composition of the elastic body;

FIG. 8 is a schematic diagram illustrating a mechanism for detecting a first recess and a second recess by a sensor unit;

FIG. 9 is a block diagram illustrating an exemplary configuration relating to determination of a load direction;

FIG. 10 is a diagram illustrating an example of a relation between patterns of a state of a plurality of grooves on the back surface of the elastic body stored as pattern image data and load directions;

FIG. 11 is a flowchart illustrating a process of determining the load direction by the detection device;

FIG. 12 is a schematic diagram illustrating an example of a relation between an assist device and the shoes;

FIG. 13 is a schematic diagram illustrating another exemplary configuration that can detect the state of a plurality of grooves included in the design provided on the back surface of the elastic body; and

FIG. 14 is a schematic diagram illustrating still another exemplary configuration that can detect the state of a plurality of grooves included in the design provided on the back surface of the elastic body.

### DETAILED DESCRIPTION

Exemplary embodiments according to the present disclosure are described below with reference to the accompanying drawings. What is disclosed herein is given by way of example only, and appropriate modifications made without departing from the spirit of the invention and easily conceivable by those skilled in the art naturally fall within the scope of the present disclosure. To further clarify the explanation, the drawings may possibly illustrate the width, the thickness, the shape, and other elements of each unit more schematically than the actual aspect. These elements, however, are given by way of example only and are not intended to limit interpretation of the present disclosure. In the present specification and the figures, components similar to those previously described with reference to previous figures are denoted by the same reference numerals, and detailed explanation thereof may be appropriately omitted.

In this disclosure, when an element is described as being “on” another element, the element can be directly on the other element, or there can be one or more elements between the element and the other element.

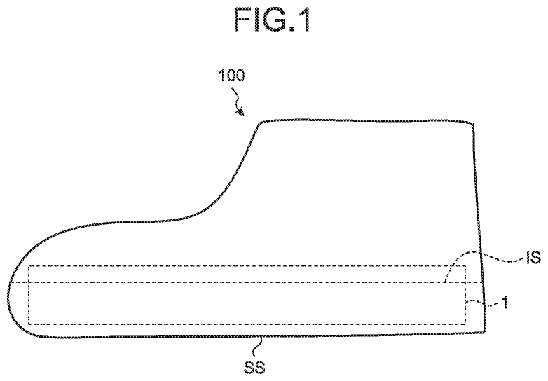

FIG. 1 is a schematic diagram of a shoe 100 including a detection device 1. The shoe 100 includes an insole IS therein. The detection device 1 is provided in the shoe 100 and is positioned on a shoe sole SS. The detection device 1 is provided with its part (design-comprising parts 10, which will be described later) overlapping the position of the insole IS with respect to the shoe sole SS.

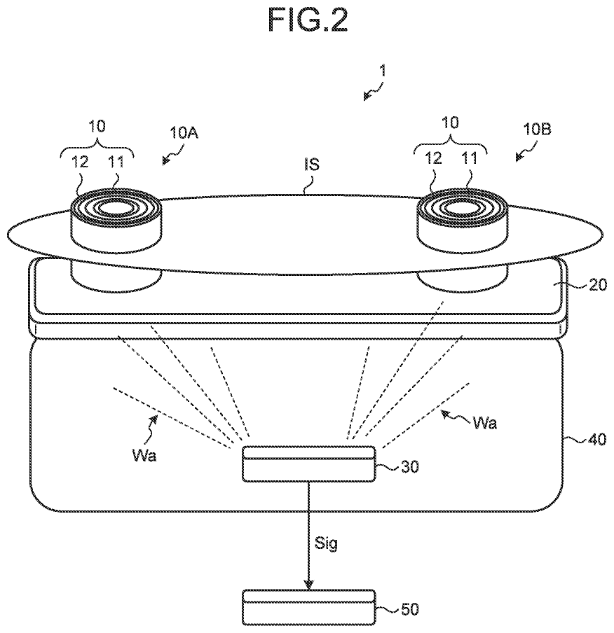

FIG. 2 is a schematic diagram illustrating an exemplary main configuration of the detection device 1. The detection device 1 includes the design-comprising parts 10, an insulator 20, a sensor unit 30, and a support member 40, for example.

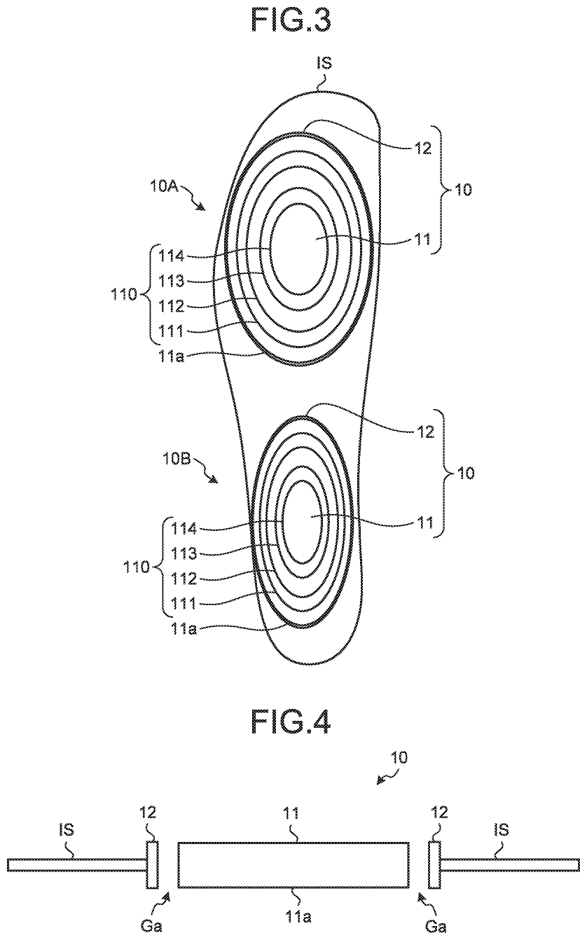

The design-comprising part 10 includes an elastic body 11 and an outer frame 12. In the example illustrated in FIG. 2 and FIG. 3, which will be described later, a design-comprising part 10A and a design-comprising part 10B are illustrated as two design-comprising parts 10 provided at different positions from the insole IS. The design-comprising part 10A and the design-comprising part 10B have the same configuration. In the following description, the term “design-comprising part 10” is used to describe the configuration common to the design-comprising part 10A and the design-comprising part 10B.

FIG. 2 is a schematic diagram illustrating only for explaining the main configuration of the detection device 1 and a basic positional relation between the design-comprising parts 10 and other components. FIG. 2 does not specifically illustrate the size of the design-comprising parts 10A and 10B and the shape of the insole IS.

FIG. 3 is a plan view illustrating an example of a specific shape of the insole IS and the difference in size between the design-comprising parts 10A and 10B. The shapes of the insole IS and the shoe 100 illustrated in FIG. 3 are the shapes of the insole IS and the design-comprising parts 10 when viewed from the shoe sole SS of the shoe 100 (refer to FIG. 1). In the example illustrated in FIG. 3, the design-comprising part 10A is larger than the design-comprising part 10B. This is, however, an example of the difference in size between the design-comprising parts 10A and 10B, and the embodiment is not limited thereto. The design-comprising part 10A and the design-comprising part 10B may have the same size, the design-comprising part 10A may be smaller than the design-comprising part 10B, or the design-comprising part 10A and the design-comprising part 10B may have different shapes.

A design 110 is formed on a surface of the elastic body 11 facing the shoe sole SS. The design 110 has a plurality of grooves, such as grooves 111, 112, 113, and 114 illustrated in FIG. 3. The grooves 111, 112, 113, and 114 are independent ring-shaped recesses. The groove 114 is positioned on the inner side of the grooves 111, 112, and 113. The groove 113 is positioned on the inner side of the grooves 111 and 112. The groove 112 is positioned on the inner side of the groove 111. The bottoms of the respective grooves included in the design 110 are recessed toward the opposite side to the insulator 20 with respect to the surface of the elastic body 11 facing the shoe sole SS. In other words, the part between the grooves 111 and 112, the part between the grooves 112 and 113, the part between the grooves 113 and 114, and the part on the inner side of the groove 114 and the part on the outer side of the groove 111 protrude toward the insulator 20 with respect to the bottoms of the grooves 111, 112, 113, and 114. In addition, these parts uniformly protrude with respect to the bottoms and form a plane. That is, the “plane” of the elastic body 11 facing the shoe sole SS is composed of parts other than the grooves included in the design 110, of parts of the elastic body 11 facing the shoe sole SS.

While FIG. 2 schematically illustrates multiple circles similar to the design 110 on the surface (front surface) opposite to the surface (back surface) of the elastic body 11 facing the insulator 20, this is given by way of schematic example only. The design 110 is not necessarily provided on the front surface. The elastic body 11 simply needs to have the design 110 on at least the back surface. The front surface of the elastic body 11 comes into contact with the sole of a foot of a user wearing the shoe 100 and deforms and moves by receiving a load from the foot.

The outer frame 12 is provided as a ring-shaped frame in planar view illustrated in FIG. 3. The outer frame 12 restricts the range in which the shape and the position of the elastic body 11 can change in deformation and movement of the elastic body 11 caused by receiving the load from the foot of the user wearing the shoe 100, within the ring-shaped frame of the outer frame 12.

FIG. 4 is a sectional view illustrating an example of the relation between the design-comprising part 10 and the insole IS. As illustrated in FIGS. 3 and 4, the outer frame 12 is interposed between the elastic body 11 and the insole IS. Specifically, as illustrated in FIG. 4, the outer frame 12 is provided as a wall member that separates the elastic body 11 from the insole IS in sectional view. The elastic body 11 is a three-dimensional elastic body, for example, a columnar elastic body provided on the inner side of the outer frame 12.

In FIG. 4 and FIGS. 5 and 6, which will be described later, a space Ga is clearly illustrated between an outer peripheral side surface 11a of the elastic body 11 and the outer frame 12. This is, however, given by way of schematic example only for indicating that the elastic body 11 is not connected to the outer frame 12. In an actual structure, a clear gap, such as the space Ga, may or may not be formed between the outer frame 12 and the elastic body 11 in a state in which no load is applied to the elastic body 11, and the outer peripheral side surface 11a may or may not be in contact with the inner peripheral surface of the outer frame 12 (refer to FIG. 3).

In FIG. 4, the outer frame 12 is provided so as to serve as a wall surface protruding toward both surfaces of the insole IS. The outer frame 12, however, simply needs to be a rigid body that defines the deformable range of the elastic body 11 and is not necessarily formed into the wall surface. Specifically, the outer frame 12 is a rigid body made of metal, alloy, or other compounds having a ring shape surrounding the elastic body 11, for example.

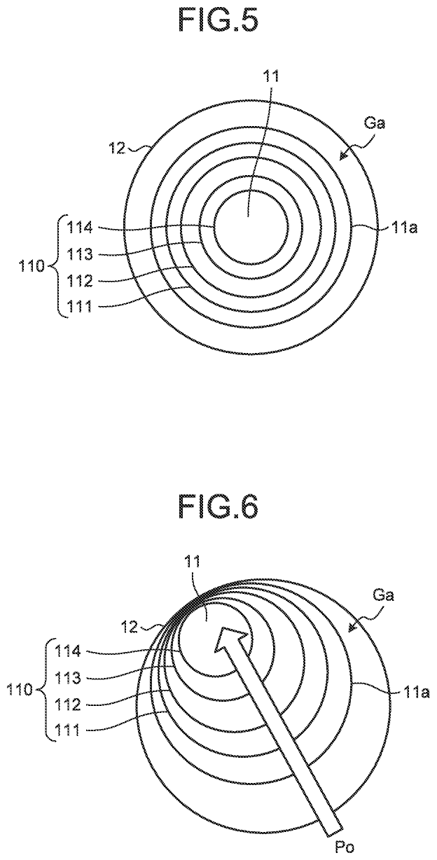

FIG. 5 is a schematic diagram illustrating the elastic body 11 in planar view in a state in which no load is applied to the elastic body 11. As illustrated in FIG. 5, a plurality of grooves (grooves 111, 112, 113, and 114) included in the design 110 are formed in the elastic body 11 so as to form concentric circles having different diameters when no load is applied to the elastic body 11 from the foot of the user wearing the shoe 100.

FIG. 6 is a schematic diagram illustrating the elastic body 11 in planar view in a state in which a load Po in a certain direction is applied to the elastic body 11. The elastic body 11 deforms such that the gaps between the grooves included in the design 110 are narrowed at parts corresponding to the direction of the load Po in the certain direction. When the direction of the load Po applied in the certain direction is represented by the positional relation between the start point and the end point (distal end) of an arrow as illustrated in FIG. 6, the gap between the grooves 111 and 112 positioned at the end point (distal end) becomes narrower than in a state in which no load is applied to the elastic body 11 (refer to FIG. 5). In addition, the gap between the grooves 112 and 113 positioned on the end point (distal end) side becomes narrower than the gap in a state in which no load is applied to the elastic body 11 (refer to FIG. 5). Similarly, the gap between the grooves 113 and 114 positioned on the end point (distal end) side becomes narrower than the gap in a state in which no load is applied to the elastic body 11 (refer to FIG. 5).

In other words, it can be determined that a load toward the side on which the gaps between the grooves included in the design 110 are narrower acts on the elastic body 11, based on the gaps between the grooves included in the design 110.

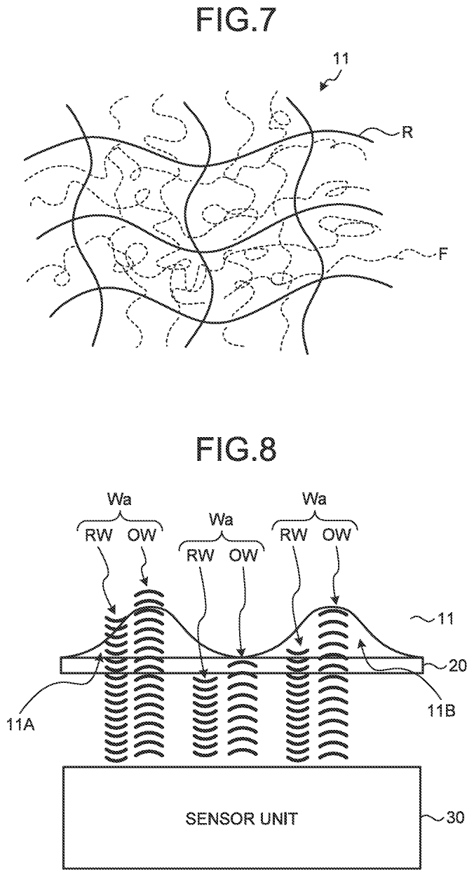

FIG. 7 is a schematic diagram illustrating a composition of the elastic body 11. The elastic body 11 contains first gel R and second gel F. The first gel R is harder than the second gel F. The second gel F is softer and more flexible than the first gel R. The elastic body 11 can deform as illustrated in FIG. 6 because it is made of a mixture of the first gel R and the second gel F. The elastic body 11 may be made of double network gel, for example.

The insulator 20 (refer to FIG. 2) is an insulator that is in contact with the back surface of the elastic body 11. The insulator 20 according to the embodiment is a thin plate member having ultrasonic permeability high enough for the sensor unit 30 to detect the state of the back surface of the elastic body 11 using ultrasonic waves. Specifically, the insulator 20 is a thin plate made of synthetic resin that never or hardly block ultrasonic waves.

The sensor unit 30 (refer to FIG. 2) is provided at a position facing the design-comprising part 10 with the insulator 20 interposed therebetween. The sensor unit 30 irradiates the back surface of the elastic body 11 with output waves OW (refer to FIG. 8). Based on its reflected waves RW (refer to FIG. 8), the sensor unit 30 generates an image that enables detecting the gaps between the grooves included in the design 110 formed on the back surface of the elastic body 11. Herein, the output waves OW and the reflected waves RW are ultrasonic waves Wa. In other words, the sensor unit 30 generates an ultrasonic image indicating the state of the back surface of the elastic body 11.

FIG. 8 is a schematic diagram illustrating a mechanism for detecting a first recess 11A and a second recess 11B by the sensor unit 30. The sensor unit 30 outputs the output waves OW to the elastic body 11 from the surface facing the elastic body 11 with the insulator 20 interposed therebetween. At least part of the output waves OW are reflected by the back surface of the elastic body 11 to be the reflected waves RW and travel toward the sensor unit 30. The reflected waves RW are detected by the sensor unit 30.

Herein, on the back surface of the elastic body 11, a plurality of grooves, such as the first recess 11A and the second recess 11B illustrated in FIG. 8 are formed. The first recess 11A and the second recess 11B are grooves included in the design 110. In the design 110 described with reference to FIGS. 3 to 6, the first recess 11A is the groove 111, 112, or 113. The second recess 11B is the groove 112, 113, or 114 and is a groove positioned on the inner side of the first recess 11A.

The ultrasonic waves Wa are attenuated as they travel in the air for a longer distance. Consequently, the ultrasonic waves Wa reflected by the part having the first recess 11A and the second recess 11B on the back surface of the elastic body 11 to be the reflected waves RW, are much more attenuated than the ultrasonic waves Wa reflected by the part having neither the first recess 11A nor the second recess 11B on the back surface of the elastic body 11 to be the reflected waves RW. The sensor unit 30 generates an ultrasonic image indicating the positions of the first recess 11A and the second recess 11B on the back surface of the elastic body 11 based on the difference in the degree of such attenuation.

The support member 40 (refer to FIG. 2) is a box-shaped member, for example. The sensor unit 30 is fixed to the inside of the support member 40. As a result, the position of the sensor unit 30 is fixed with respect to the outer frame 12. The sensor unit 30 irradiates the elastic body 11 with the output waves OW in a manner covering at least the inside of the outer frame 12, thereby detecting the reflected waves RW from the elastic body 11 and generating the ultrasonic image described above. The sensor unit 30 may be fixed to the support member 40 by any desired method. Examples of the method include, but are not limited to, adhesion, screwing, fitting, etc.

The insulator 20 and the support member 40 are fixed on the shoe sole SS in the shoe 100. Specifically, the support member 40 and the insulator 20 are embedded and fixed in a space formed in the shoe 100 in the order as listed. As a result, the positional relation between the insulator 20 and the support member 40 is defined. The outer frame 12 is fixed to the insole IS. Disposing the insole IS in the shoe 100 defines the position of the outer frame 12. The elastic body 11 is deformably formed in the outer frame 12.

The insulator 20 and the support member 40 receive force from above due to the weight or the like of the user wearing the shoe 100. Consequently, the insulator 20 and the support member 40 preferably have strength high enough to withstand the force. Specifically, the insulator 20 and the support member 40 are provided as structures made of such material and having such a thickness that have the strength, for example. The part of the support member 40 facing the insulator 20 may be opened or closed with a lid-shaped structure having a thickness thin enough not to prevent the output waves OW and the reflected waves RW from propagating.

A determiner 50 (refer to FIG. 2) determines a load direction of the load applied to the elastic body 11 based on an ultrasonic image generated by the sensor unit 30.

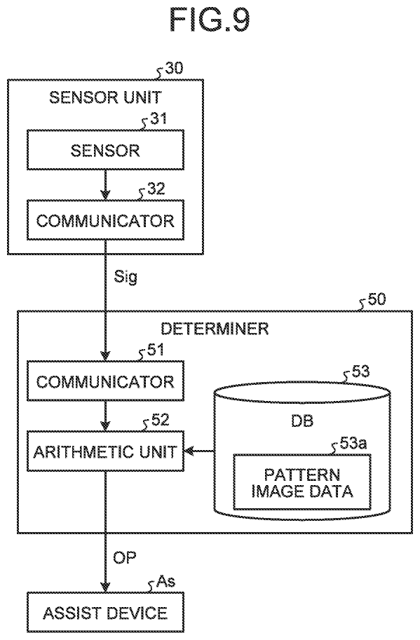

FIG. 9 is a block diagram illustrating an exemplary configuration relating to determination of a load direction. The sensor unit 30 includes a sensor 31 and a communicator 32, for example. The sensor 31 has functions relating to inputting/outputting and detecting the ultrasonic waves Wa described above and generating an ultrasonic image, out of the functions of the sensor unit 30. In other words, the sensor 31 outputs the output waves OW, detects the reflected waves RW, and generates an ultrasonic image. The communicator 32 transmits the ultrasonic image generated by the sensor 31 to the determiner 50. Specifically, the communicator 32 includes a circuit or the like functioning as a network interface controller (NIC) and communicates with an external information processing device. The communicator 32 transmits data of the ultrasonic image in accordance with a predetermined protocol. FIGS. 2 and 9 illustrate transmission of data of the ultrasonic image by illustrating a signal Sig generated by transmission of data performed by the communicator 32.

The determiner 50 includes a communicator 51, an arithmetic unit 52, and a database (DB) 53, for example. The communicator 51 receives data of an ultrasonic image transmitted by the communicator 32. Specifically, the communicator 51 includes a circuit or the like functioning as a NIC that performs communications in accordance with the same predetermined protocol as that of the communicator 32.

The arithmetic unit 52 is an information processing device including a central processing unit (CPU), a semiconductor memory, a non-volatile memory, or the like for example. The semiconductor memory temporarily stores therein software programs, data, parameters, and the like used in an arithmetic operation performed by the CPU. The non-volatile memory stores therein the software programs. The arithmetic unit 52 may be a single integrated circuit that functions similarly to the information processing device. Examples of the integrated circuit include, but are not limited to, field programmable gate array (FPGA), application specific integrated circuit (ASIC), etc. The integrated circuit is not limited thereto and may be provided as another aspect.

The arithmetic unit 52 acquires data of an ultrasonic image received by the communicator 51. The arithmetic unit 52 performs determination processing of determining the direction of a load applied to the elastic body 11 at the moment when the state of the elastic body 11 corresponding to the ultrasonic image is detected by the sensor unit 30, based on the ultrasonic image. The arithmetic unit 52 refers to the DB 53 to perform the determination processing.

The DB 53 stores therein pattern image data 53a. The pattern image data 53a is image data indicating the state of the back surface of the elastic body 11 when a load corresponding to a predetermined load direction is applied thereto. More specifically, the pattern image data 53a is image data indicating the state of the gaps between the grooves included in the design 110 that changes depending on the load direction.

The DB 53 associates information indicating a load direction with the pattern image data 53a corresponding to the load direction.

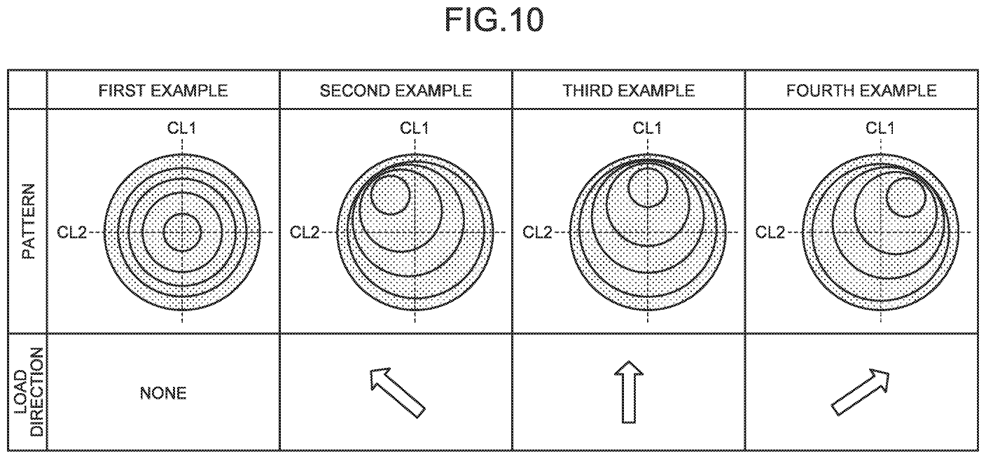

FIG. 10 is a diagram illustrating an example of the relation between the patterns of the state of the grooves on the back surface of the elastic body 11 stored as the pattern image data 53a and the load directions. In the following description with reference to FIG. 10, the term “pattern” refers to a pattern of the state of the grooves on the back surface of the elastic body 11 stored as the pattern image data 53a. In each of a plurality of “patterns” illustrated in FIG. 10, a dashed line CL1 and a dashed line CL2 are illustrated. The dashed line CL1 is orthogonal to the dashed line CL2. The intersection of the dashed lines CL1 and CL2 corresponds to the center of the circles formed by the grooves included in the design 110 when no load is applied to the elastic body 11.

The outermost circles of the respective “patterns” illustrated in FIG. 10 correspond to the outer peripheral side surface 11a. The circles on the inner side of the outermost circle correspond to the grooves included in the design 110.

In the “pattern” of the “first example” illustrated in FIG. 10, the grooves form concentric circles around the intersection of the dashed lines CL1 and CL2. This pattern corresponds to the state of the elastic body 11 in a state in which no load is applied thereto as described with reference to FIG. 5. Consequently, the “load direction” corresponding to the “pattern” of the “first example” is “none”.

In the “patterns” of the “second example”, the “third example”, and the “fourth example” illustrated in FIG. 10, the gaps between the grooves are narrowed at different positions, respectively, and the gaps between the grooves on the side opposite to the position with the intersection of the dashed lines CL1 and CL2 therebetween are relatively widened compared with the position. These patterns correspond to the state of the elastic body 11 when a load is applied thereto as described with reference to FIG. 6. Consequently, the “load directions” corresponding to the “patterns” of the “second example”, the “third example”, and the “fourth example” are directions toward the part in which the gaps between the grooves are narrowed from the side opposite to the part with the intersection of the dashed lines CL1 and CL2 therebetween.

The pattern image data 53a in the DB 53 is data of a pattern image identical with or similar to the pattern corresponding to any one of the “patterns” illustrated in FIG. 10, for example. The term “similar” referred to herein is, for example, a state in which a difference between a detected pattern, that is, a pattern formed by the grooves on the back surface of the elastic body 11 detected by the sensor unit 30 and a pattern formed by the positions of the circles in the pattern image data falls within a predetermined range. The predetermined range is set based on the size and the shape of the grooves of the elastic body 11 in designing the elastic body 11. The DB 53 stores therein not only the pattern image data 53a like the “patterns” illustrated in FIG. 10, but also the pattern image data 53a corresponding to a larger number of load directions than the directions of the load that can actually act on the elastic body 11. The DB 53 also stores therein information indicating the load directions associated with the respective stored pattern image data 53a.

When acquiring data of an ultrasonic image from the communicator 51, the arithmetic unit 52 refers to one of the pattern image data 53a stored in the DB 53 and performs pattern matching processing of comparing the ultrasonic image with the image of the pattern image data 53a. In the pattern matching processing, the ultrasonic image is defined as a first image, and the image of the pattern image data 53a is defined as a second image. If the first image and the second image are identical with or similar to each other, the arithmetic unit 52 determines the load direction associated with the image (second image) of the pattern image data 53a in the DB 53 to be the load direction applied to the elastic body 11 at the moment when the state of the elastic body 11 corresponding to the ultrasonic image (first image) is detected by the sensor unit 30. On the other hand, if the first image and the second image are neither identical with nor similar to each other, the arithmetic unit 52 refers to another one of the pattern image data 53a stored in the DB 53 and performs pattern matching processing again. The arithmetic unit 52 repeatedly performs pattern matching processing until the pattern image data 53a (second image) identical with or similar to the first image is discovered and the load direction is identified.

The detailed processing of determining whether the first image and the second image are identical with or similar to each other in pattern matching processing may be based on a typical image pattern matching algorithm or an algorithm designed specifically for pattern matching processing. Examples of the typical image pattern matching algorithm include, normalized cross correlation (NCC), zero-mean NCC (ZNCC), sum of squared difference (SSD), sum of absolute difference (SAD), etc.

To further improve the accuracy in pattern matching processing, the pattern image data 53a according to the embodiment is preferably ultrasonic image data. In other words, the pattern image data 53a is preferably ultrasonic image data generated and registered in the DB 53 by the sensor unit 30 that actually detects the elastic body 11 that receives the load corresponding to the load direction associated therewith in the DB 53.

As schematically illustrated in FIG. 10, the elastic body 11 has a surface part on which the design 110 including the first recess 11A and the second recess 11B is formed. The elastic body 11 forms a first design generated on the surface part by the first recess 11A and the second recess 11B according to a first load and a second design generated on the surface part by the first recess 11A and the second recess 11B according to a second load. The first design is different from the second design. Herein, the “designs” are schematically illustrated as the “patterns” in FIG. 10. The “designs” are generated by the first recess 11A and the second recess 11B, that is, by the designs 110 and can be detected by the sensor unit 30 as described with reference to FIG. 8. The “first load” and the “second load” are schematically illustrated by the “load directions” in FIG. 10. The larger the force applied to the elastic body 11 as a load, the higher the density of the grooves in the load direction. The “density of the grooves” refers to the narrowness of the gaps between the grooves included in the design 110. Consequently, “the density of the grooves being higher” refers to the state where the gaps between the grooves included in the design 110 are narrower. Thus, the detection device 1 may prepare a plurality of pattern images having different densities of the grooves depending on the magnitude of the load to be included in the pattern image data 53a and associate them with information indicating the magnitude of the load. As a result, the detection device 1 can further determine the magnitude of the load. The detection device 1 may add the information indicating the magnitude of the load determined in this manner to output information OP, which will be described later, to enable an assist device As to perform an operation corresponding to the magnitude of the load.

The functions of the sensor unit 30 and the determiner 50 may be provided in an integrated device. In this case, the communicators 32 and 51 are omitted, and data of the ultrasonic image output from the sensor 31 is input to the arithmetic unit 52. The DB 53 may be provided to another device different from the determiner 50. In this case, the other device is provided with a communicator similar to the communicator 51. The other device communicates with the determiner 50, thereby enabling the arithmetic unit 52 to refer to the DB 53.

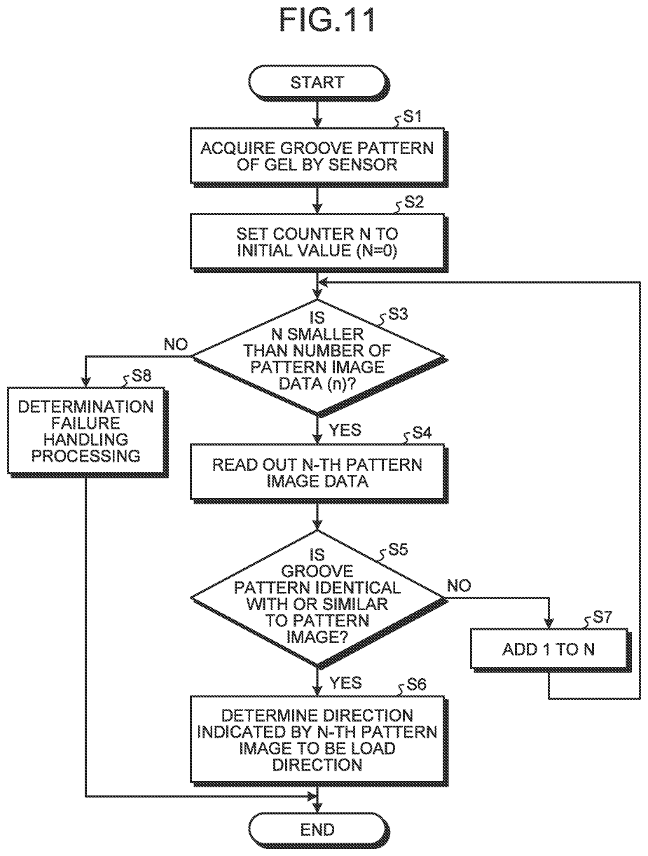

FIG. 11 is a flowchart illustrating a process of determining the load direction by the detection device 1. First, the sensor unit 30 acquires a groove pattern of the gel (Step S1). The groove pattern of the gel referred to herein indicates a pattern formed by the grooves included in the design 110 provided on the back surface of the elastic body 11. Specifically, the sensor 31, for example, irradiates the back surface of the elastic body 11 with the output waves OW and detects the reflected waves RW, thereby generating an ultrasonic image indicating the state of the grooves included in the design 110 formed on the back surface of the elastic body 11 in the processing at Step S1. The communicator 32 transmits data of the ultrasonic image generated by the sensor 31 to the determiner 50. The arithmetic unit 52 acquires the data of the ultrasonic image generated by the sensor 31 via the communicator 51.

The arithmetic unit 52 sets a counter for controlling the progress of pattern matching processing. Specifically, the arithmetic unit 52 sets an initial value of a variable N used in the counter to 0 (N=0) (Step S2).

The arithmetic unit 52 determines whether the value of N is smaller than the number of pattern image data 53a stored in the DB 53. Specifically, when the number of pattern image data 53a stored in the DB 53 is n, the arithmetic unit 52 determines whether N<n is satisfied (Step S3). If the arithmetic unit 52 determines that N<n is satisfied (Yes at Step S3), the arithmetic unit 52 reads out the N-th pattern image data 53a stored in the DB 53 (Step S4). In the processing at Step S4, the arithmetic unit 52 handles the zero-th pattern image data 53a as the first pattern image data 53a.

The arithmetic unit 52 determines whether the groove pattern acquired in the processing at Step S1 is identical with or similar to the groove pattern indicated by the pattern image data 53a acquired in the processing at Step S4 performed immediately before this Step S5 (Step S5). If the arithmetic unit 52 determines that the groove pattern acquired in the processing at Step S1 is identical with or similar to the groove pattern indicated by the pattern image data 53a acquired in the processing at Step S4 performed immediately before this Step S5 (Yes at Step S5), the arithmetic unit 52 determines the direction indicated by the N-th pattern image data 53a to be the load direction (Step S6). In other words, the arithmetic unit 52 acquires information indicating the load direction associated with the N-th pattern image data 53a in the DB 53 and determines the load direction to be the direction indicated by the N-th pattern image data 53a and to be information on the load applied to the elastic body 11 at the timing when the processing at Step S1 is performed. The arithmetic unit 52 may also determine the magnitude of the load at Step S6 based on the determination of identicalness or similarity at Step S5.

If, at Step S5, the arithmetic unit 52 determines that the groove pattern acquired in the processing at Step S1 is neither identical with nor similar to the groove pattern indicated by the pattern image data 53a acquired in the processing at Step S4 performed immediately before this Step S5 (No at Step S5), the arithmetic unit 52 adds 1 to the value of N (Step S7). After Step S7, the arithmetic unit 52 performs the processing at Step S3 again.

If the arithmetic unit 52 determines that N<n is not satisfied, that is, N=n is satisfied in the processing at Step S3 (No at Step S3), the arithmetic unit 52 performs determination failure handling processing that is set in advance (Step S8). The determination failure handling processing may be arbitrary processing. The determination failure handling processing may be processing of outputting an error indicating that the load direction fails to be detected, processing of deferring determination of the load direction until the processing at Step S6 performed after the processing at Step S1 performed again after Step S8, or other processing.

The embodiment repeats detecting the state of the back surface of the elastic body 11, generating an ultrasonic image based on the detection, and outputting the ultrasonic image by the sensor unit 30, and determination processing based on the ultrasonic image by the arithmetic unit 52, every time a predetermined time has elapsed. Consequently, it is possible to continue to periodically determine the load direction applied by the user wearing the shoe 100 to the elastic body 11, that is, to the shoe sole provided with the insole IS at predetermined time intervals.

As described above, the detection device 1 according to the embodiment includes the elastic body 11 and the sensor (sensor unit 30). The elastic body 11 has a surface part on which the first recess 11A and the second recess 11B are formed. The elastic body 11 forms a first design generated on the surface part by the first recess 11A and the second recess 11B according to a first load and a second design generated on the surface part by the first recess 11A and the second recess 11B according to a second load. The first design is different from the second design. The sensor (sensor unit 30) detects recesses and protrusions on the surface part. With this configuration, the detection device 1 can determine the load direction applied to the elastic body 11 based on the design generated by the detected recesses and protrusions on the detected surface part. Consequently, the detection device 1 is suitable for determining the load direction applied to an object (e.g., a shoe sole) by an acting subject (e.g., a user who is a person).

The first recess 11A and the second recess 11B are independent ring-shaped recesses. The second recess 11B is positioned on the inner side of the first recess 11A. Consequently, the detection device 1 can determine the load direction based on a change in the gap between the circle formed by the first recess 11A and the circle formed by the second recess 11B.

The detection device 1 further includes the determiner 50 that determines that the elastic body 11 is receiving a load acting toward the side on which the gap between the first recess 11A and the second recess 11B is narrower. Consequently, the detection device 1 can complete determining the load direction.

The elastic body 11 is provided so as to deform within a deformable range defined by the outer frame 12 provided surrounding the elastic body 11. The sensor unit 30 is fixed to a predetermined position. The deformable range of the elastic body 11 according to the embodiment is on the inner side of the outer frame 12. The sensor unit 30 is fixed to the support member 40. Consequently, the detection device 1 can perform detection by the sensor unit 30 more accurately.

The surface (front surface) of the elastic body 11 opposite to the surface part (back surface) on which the first recess 11A and the second recess 11B are formed comes into contact with the sole of a foot of the user wearing the shoe 100. The sensor unit 30 is provided closer to the shoe sole SS than the elastic body 11 is, in the shoe 100. Consequently, the detection device 1 can determine the direction of a load applied to the shoe 100 by movement, such as walking, of the user wearing the shoe 100.

The sensor 31 is an ultrasonic sensor that transmits the output waves OW of the ultrasonic waves Wa to the elastic body 11 and detects the reflected waves RW. Consequently, the detection device 1 can satisfactorily acquire the design generated by the first recess 11A and the second recess 11B.

The detection device 1 may transmit, to the assist device As, the output information OP (refer to FIG. 9) indicating the load direction derived at determination processing performed by the arithmetic unit 52. The assist device As assists the user wearing the shoe 100 in walking.

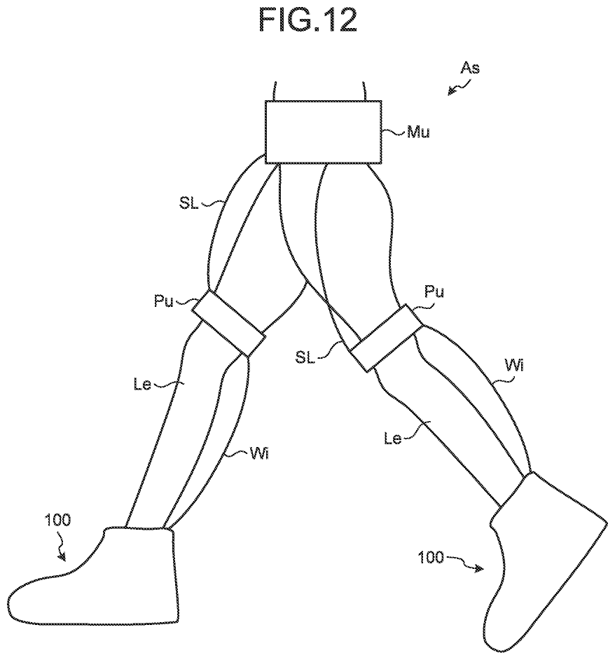

FIG. 12 is a schematic diagram illustrating an example of the relation between the assist device As and the shoes 100. The assist device As includes a waist unit Mu, leg units Pu, and connections SL, for example. The assist device As assists movement of the leg units Pu fixed to respective legs Le of the user by an actuator, such as a motor, provided to the waist unit Mu. The leg units Pu are connected to the motor by the respective connections SL. Wiring Wi extends from the shoe 100 illustrated in FIG. 12. In the shoe 100 illustrated in FIG. 12, the sensor unit 30 and the determiner 50 are provided in the support member 40, or the determiner 50 is integrated with the sensor unit 30. The wiring Wi transmits the output information OP (refer to FIG. 9) output from the arithmetic unit 52 of the determiner 50. The signal transmission path serving as the wiring Wi continuously extends to the waist unit Mu through the connection SL. A control circuit, which is not illustrated, provided to the waist unit Mu grasps the direction of movement, that is, the state of walking of the user based on the load direction indicated by the output information OP. The control circuit operates the actuator based on the state of walking grasped in this manner, thereby assisting the user in walking.

The walking support system described with reference to FIG. 12 includes the detection device 1 and the assist device As that assists the user in walking. The detection device 1 outputs information (output information OP) indicating a load direction. The load direction is determined based on the first recess 11A and the second recess 11B detected by the sensor unit 30. As a result, the detection device 1 can supply the assist device As with the information indicating the load direction determined based on the design generated by the first recess 11A and the second recess 11B due to walking of the user. Consequently, the assist device As can assist walking more accurately based on the information indicating the load direction.

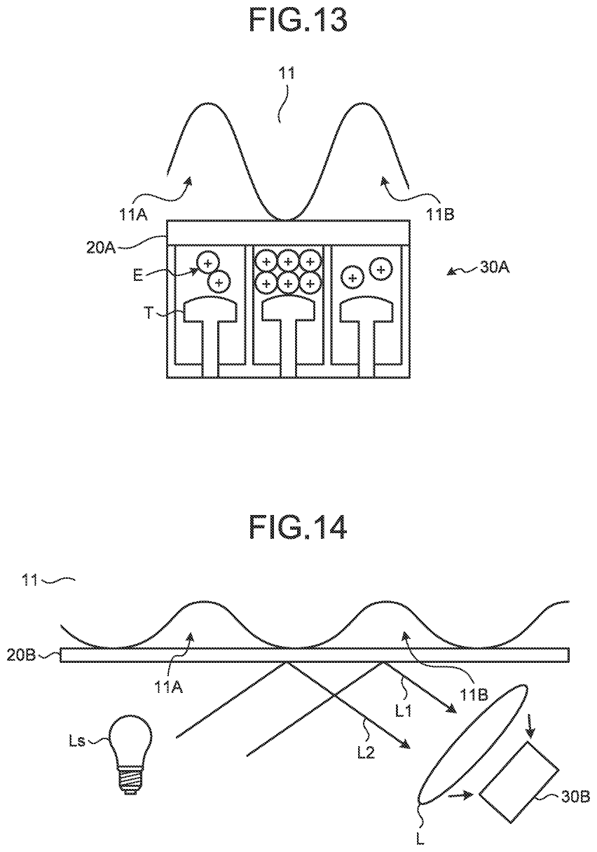

While the sensor unit 30 generates an ultrasonic image using the ultrasonic waves Wa, thereby detecting the state of the grooves included in the design 110 provided on the back surface of the elastic body 11, the configuration that can detect the state of the grooves is not limited thereto. The following describes other exemplary configurations that can detect the state of the grooves with reference to FIGS. 13 and 14.

FIG. 13 is a schematic diagram illustrating another exemplary configuration that can detect the state of the grooves included in the design 110 provided on the back surface of the elastic body 11. A sensor 30A illustrated in FIG. 13 is a touch sensor (capacitive sensor) that detects whether the elastic body 11 comes into contact with a detection surface 20A based on capacitance. Specifically, the sensor 30A includes therein a plurality of detection electrodes T. Electric charges E between the detection electrodes T and the detection surface 20A greatly change depending on whether the elastic body 11 disposed at a position facing the detection electrodes T with the detection surface 20A interposed therebetween is in contact with the detection surface 20A. The grooves, such as the first recess 11A and the second recess 11B, do not come into contact with the detection surface 20A. By contrast, the back surface of the part of the elastic body 11 on which neither the first recess 11A nor the second recess 11B is formed comes into contact with the detection surface 20A. The detection surface 20A is disposed at the position of the insulator 20, and the detection electrodes T are disposed so as to cover the range of contact between the detection surface 20A and the elastic body 11, whereby the sensor 30A can two-dimensionally detect the positions of the grooves included in the design 110 provided on the back surface of the elastic body 11. Consequently, as the sensor unit 30 generates an ultrasonic image, the sensor 30A can generate a two-dimensional image indicating the positions of the grooves.

FIG. 14 is a schematic diagram illustrating still another exemplary configuration that can detect the state of the grooves included in the design 110 provided on the back surface of the elastic body 11. A sensor 30B is an optical sensor that detects light (e.g., light L1 and light L2) emitted from a light source Ls, refracted by a detection surface 20B, and guided by a lens L. The detection surface 20B is made of light-transmitting resin or glass. Light is refracted more clearly at a boundary between objects having different refractive indexes. The refractive index of the elastic body 11 is different from that of the air between the grooves, such as the first recess 11A and the second recess 11B. As a result, a refractive index at a position where the back surface of the elastic body 11 is not in contact with the detection surface 20B because of the first recess 11A and the second recess 11B are different from a refractive index at a position where the back surface of the elastic body 11 is in contact with the detection surface 20B. Consequently, there is a difference in refractive index between the light L1 emitted from the light source Ls to the positions of the first recess 11A and the second recess 11B and the light L2 emitted from the light source Ls to the position at which the back surface of the elastic body 11 is in contact with the detection surface 20B. The difference in refractive index corresponds to the difference in intensity between the light L1 and the light L2 within the range of being detected by the sensor 30B. The sensor 30B can two-dimensionally detect the positions of the grooves included in the design 110 provided on the back surface of the elastic body 11 based on the intensity of light.

While the elastic body 11 is made of gel in the example described above, it may be other elastic members, such as rubber. While the wall formed by the outer frame 12 illustrated in FIG. 4 protrudes toward both surfaces of the insole IS, it may protrude toward one of the surfaces. The wall formed by the outer frame 12, for example, may be prevented from protruding on one surface of the insole IS with which the foot of the user wearing the shoe 100 comes into contact. This structure makes the shoe 100 more comfortable to wear. Alternatively, the wall formed by the outer frame 12 may be prevented from protruding on the other surface of the insole IS facing the insulator 20. This structure can bring the insole IS into contact with the insulator 20, and a component for filling the space between the insole IS and the insulator 20 can be omitted. The component for filling the space between the insole IS and the insulator 20 is preferably a member selected considering comfortability of the shoe 100 to wear and reduction in load to the insulator 20, such as cushioning material.

While the detection device 1 is provided in the shoe 100 in the example described above, the embodiment is not limited thereto. The detection device 1 may be provided between a component that vibrates, such as a machine including a prime motor, and a floor or the ground. In this case, the detection device 1 can detect the presence of vibration and the load direction due to the vibration based on a change in the positions of the grooves included in the design 110 provided on the back surface of the elastic body 11.

The grooves included in the design 110 need not be complete circles. The grooves simply need to have such a shape and be disposed at such positions that enable determining the load direction based on displacement in the positional relation. The first recess 11A and the second recess 11B, for example, may each have one or more arcs forming a circular design. Alternatively, the first recess 11A and the second recess 11B may be not circular but polygonal grooves.

Out of other advantageous effects provided by the aspects described in the present embodiment, advantageous effects clearly defined by the description in the present specification or appropriately conceivable by those skilled in the art are naturally provided by the present disclosure.

What is claimed is:

1. A detection device comprising:
an elastic body that has a surface part on which a first recess and a second recess are formed and that forms a first design generated on the surface part by the first recess and the second recess according to a first load and a second design generated on the surface part by the first recess and the second recess according to a second load, the first design being different from the second design; and
a sensor configured to detect a recess and a protrusion on the surface part.

2. The detection device according to claim 1, wherein
the first recess and the second recess are independent ring-shaped recesses, and
the second recess is positioned on an inner side of the first recess.

3. The detection device according to claim 1, further comprising a determiner configured to determine that the elastic body is receiving a load acting toward a side on which a gap between the first recess and the second recess is narrower.

4. The detection device according to claim 1, further comprising an insulator provided between the elastic body and the sensor.

5. The detection device according to claim 1, wherein
the elastic body is provided so as to deform within a deformable range defined by an outer frame provided surrounding the elastic body, and
the sensor is fixed to a predetermined position.

6. The detection device according to claim 1, wherein
a part of the elastic body opposite to the surface part comes into contact with a sole of a foot of a user wearing a shoe, and
the sensor is provided closer to a shoe sole than the elastic body is, in the shoe.

7. The detection device according to claim 1, wherein the sensor is an ultrasonic sensor configured to transmit an ultrasonic wave to the surface part and detect a reflected wave of the ultrasonic wave.

8. The detection device according to claim 1, wherein the sensor is a capacitive sensor.

9. The detection device according to claim 1, wherein the sensor is an optical sensor.

10. The detection device according to claim 1, further comprising a determiner configured to determine that the elastic body is receiving a larger load as a gap between the first recess and the second recess is narrower.

11. A walking support system comprising:
the detection device according to claim 1; and
an assist device configured to assist a user in walking, wherein
the detection device outputs information indicating a load direction, and
the load direction is determined based on a recess and a protrusion on the surface part detected by the sensor.
